(19) United States
(12) Patent Application Publication
(10) Pub. No.: US 2012/0071980 A1
(43) Pub. Date: Mar. 22, 2012

(54) STEERABLE SPINE IMPLANT AND SYSTEM

(75) Inventors: Thomas PURCELL, Del Mar, CA (US); Morgan LORIO, Carlsbad, CA (US)

(73) Assignee: Alphatec Spine, Inc., Carlsbad, CA (US)

(21) Appl. No.: 13/218,098

(22) Filed: Aug. 25, 2011

Related U.S. Application Data
(60) Provisional application No. 61/383,582, filed on Sep. 16, 2010.

(51) Int. Cl. A61F 2/44

(52) U.S. Cl. 623/17.16

(57) ABSTRACT

Embodiments of the invention being disclosed are directed to a spine implant that allows for in situ adjustment or steering during implantation which allows for precise placement. The structure of the device is composed of a series of hinged link components connected by dowel or shear pins allowing for the links to rotate with respect to each other. The steering feature of the device is activated by a series of tension members connected or coupled to the links. As the tension members are placed in tension, typically by pulling the appropriate member, forces are placed on the individual links to actuate/rotate them in a clockwise or counterclockwise direction. By controlling the rotation of the links, the device may be steered in the desired direction.

This application claims priority to U.S. Provisional Application 61/383,582, filed Sep. 16, 2010.

### FIELD OF THE INVENTION

The present invention is directed to systems, methods, and devices applicable to surgery. More specifically, the present invention is directed to a steerable spine implant and system for use by medical personnel (i.e., doctor) in spinal and other surgical procedures.

### BACKGROUND OF THE INVENTION

Vertebrae are the individual irregular bones that make up the spinal column—a flexuous and flexible column. There are normally thirty-three vertebrae in humans, including the five that are fused to form the sacrum (the others are separated by intervertebral discs) and the four coccygeal bones which form the tailbone. The upper three regions comprise the remaining 24, and are grouped under the names cervical (7 vertebrae), thoracic (12 vertebrae) and lumbar (5 vertebrae), according to the regions they occupy. This number is sometimes increased by an additional vertebra in one region, or it may be diminished in one region, the deficiency often being supplied by an additional vertebra in another. The number of cervical vertebrae is, however, very rarely increased or diminished.

A typical vertebra consists of two essential parts: an anterior (front) segment, which is the vertebral body; and a posterior part—the vertebral (neural) arch—which encloses the vertebral foramen. The vertebral arch is formed by a pair of pedicles and a pair of laminae, and supports seven processes, four articular, two transverse, and one spinous, the latter also being known as the neural spine.

When the vertebrae are articulated with each other, the bodies form a strong pillar for the support of the head and trunk, and the vertebral foramina constitute a canal for the protection of the medulla spinalis (spinal cord), while between every pair of vertebrae are two apertures, the intervertebral foramina, one on either side, for the transmission of the spinal nerves and vessels.

Conventional interbody implants are used in spinal fusion procedures to repair damaged or incorrectly articulating vertebrae. These implants are typically rigid and are inserted between vertebrae in a straight manner or direct approach.

In some cases, the direct approach to the spine may be difficult and the current technologies do not allow for in situ steering of a spine implant. There exists a need for further improvements in the field of spine implants of the present type that may be steerable.

### SUMMARY OF THE INVENTION

Embodiments of the invention being disclosed are directed to a spine implant that allows for in situ adjustment or steering during implantation which allows for precise placement. The steerable spine implant includes a plurality of links rotatably coupled together at a hinge point including a proximal link, one or more intermediate links, and a distal link; and a plurality of tension members coupled to the intermediate and distal links, each link being coupled with first and second tension members on opposite sides of the hinge point. Activating the first tension member rotates the plurality of links in a clockwise direction and activating the second tension member rotates the plurality of links in a counterclockwise direction.

In other features, the links further include a central channel sized to carry the plurality of tension members. The tension members exit a channel at a proximal portion of the proximal link. The links further include a top portion and a bottom portion having a plurality of protrusions or teeth. The protrusions are configured to prevent movement of the steerable implant once the steerable implant is implanted. The proximal link includes one or more adapter features or apertures configured to couple to one or more instruments. One or more of the plurality of links includes openings for a graft or a DBM. The distal link includes a tapered or shaped portion configured for insertion into a spinal area. Activating the first or second tension member comprises pulling the tension member proximally. At least one of the plurality of links includes at least one of a varying height, length, thickness, and lordosis angle. The plurality of links of the steerable implant comprises a biologically inert material. Two or more of the plurality of links comprise two or more different biologically inert materials.

A steerable spine implant system includes a guide wire and a plurality of links rotatably coupled together at a hinge point including a proximal link, one or more intermediate links, and a distal link, each of the plurality of links include an aperture sized to slide over the guide wire, wherein when the plurality of links are coupled together, the apertures form a continuous guide wire channel from a proximal end to a distal end of the implant.

In other features, the links further include a top portion and a bottom portion having a plurality of protrusions or teeth. The protrusions are configured to prevent movement of the steerable implant once the steerable implant is implanted. The proximal link includes one or more adapter features or apertures configured to couple to one or more instruments. One or more of the plurality of links includes openings for a graft or a DBM. The distal link includes a tapered or shaped portion configured for insertion into a spinal area. At least one of the plurality of links includes at least one of a varying height, length, thickness, and lordosis angle. The plurality of links of the steerable implant comprise a biologically inert material.

Further features and advantages of the invention, as well as structure and operation of various embodiments of the invention, are disclosed in detail below with references to the accompanying drawings.

### BRIEF DESCRIPTION OF THE DRAWINGS

The present invention is described with reference to the accompanying drawings. In the drawings, like reference numbers indicate identical or functionally similar elements.

FIG. 1 is perspective view showing one embodiment of a steerable spine implant.

FIG. 2 is a side view of the implant shown in FIG. 1.

FIGS. 3A and 3B are sectional views of the implant shown in FIG. 1.

FIGS. 4, 5A, and 5B show one embodiment of an instrument that may be used to steer the steerable implant shown in FIG. 1.

FIGS. 6A and 6B are sectional views illustrating steering of the implant shown in FIG. 1 using the instrument shown in FIGS. 4, 5A, and 5B.

FIGS. 7A and 7B illustrate a steerable spine implant being steered into a space between vertebrae.

FIGS. 8-11B illustrate another embodiment of the steerable implant that travels along a guide wire.

### DETAILED DESCRIPTION OF THE INVENTION

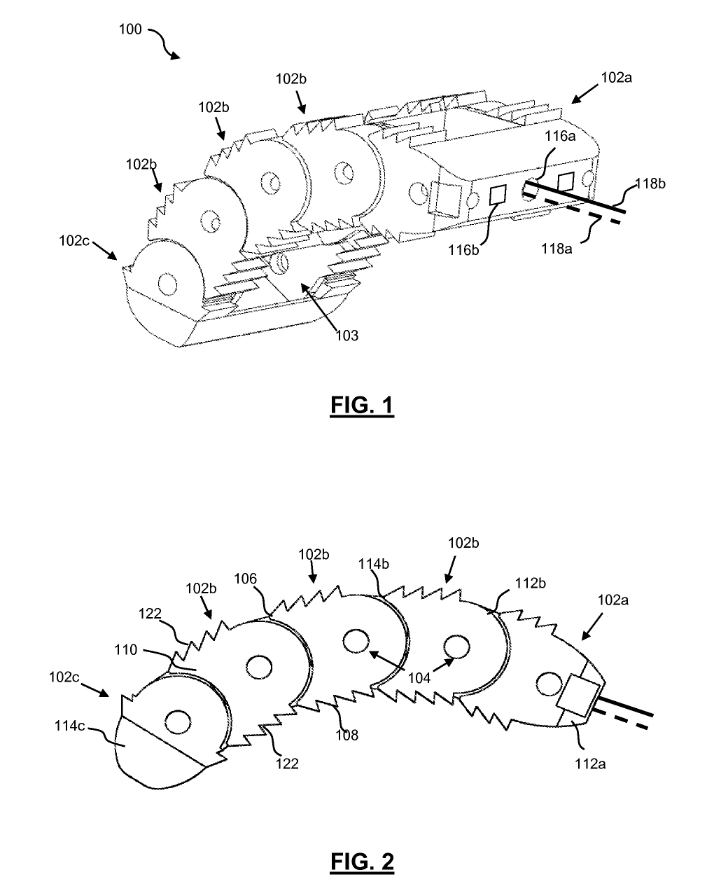

FIG. 1 is a perspective view and FIG. 2 is a side view showing one embodiment of a steerable spine implant 100 having a number of hinged links 102, including a proximal link 102a, one or more intermediate links 102b, and a distal link 102c. The links 102 are configured to rotatably link together. In some embodiments, proximal and distal end portions of the links have hinge features that interdigitate with each other and are held together with dowel or shear pins 104. This allows each of the links 102 to rotate with respect to each other. A plurality of tension members 118 are coupled to the links 102, such that pulling of the tension members rotates the links either clockwise or counterclockwise to steer the implant 100.

The implant 100 may be sized for use in many areas of the spine and may have varying height, length, thickness, and/or lordosis angle. In addition, each of the links 102 may have varying height, length, thickness, and/or lordosis angle. Links 102 may be added or removed to alter the length or configuration of the implant 100.

Each of the links 102 includes a top portion 106, a bottom portion 108, side portions 110, a proximal portion 112, and a distal portion 114. As discussed above, there are at least three different types of links: the proximal link 102a, one or more intermediate links 102b, and a distal link 102c.

The proximal portion 112a of the proximal link 102a includes one or more adapter features or apertures 116 which can be configured as attachment points for instrumentation. The apertures may be threaded circular apertures 116a for attachment to an insertion instrument. There may also be other shaped apertures 116b used for other attachment features. The distal portion 114a is configured to rotatably couple with the proximal portion 112b of intermediate link 102b. Each of the intermediate links 102b are rotatably coupled together, with the distal portions 114b being coupled with the proximal portions 112b. The distal link 102c has a proximal portion 112c coupled to the distal portion 114b of the adjacent intermediate link 102b. The distal portion 114c of the distal link 102c may include a tapered or shaped portion configured for insertion into the spinal area.

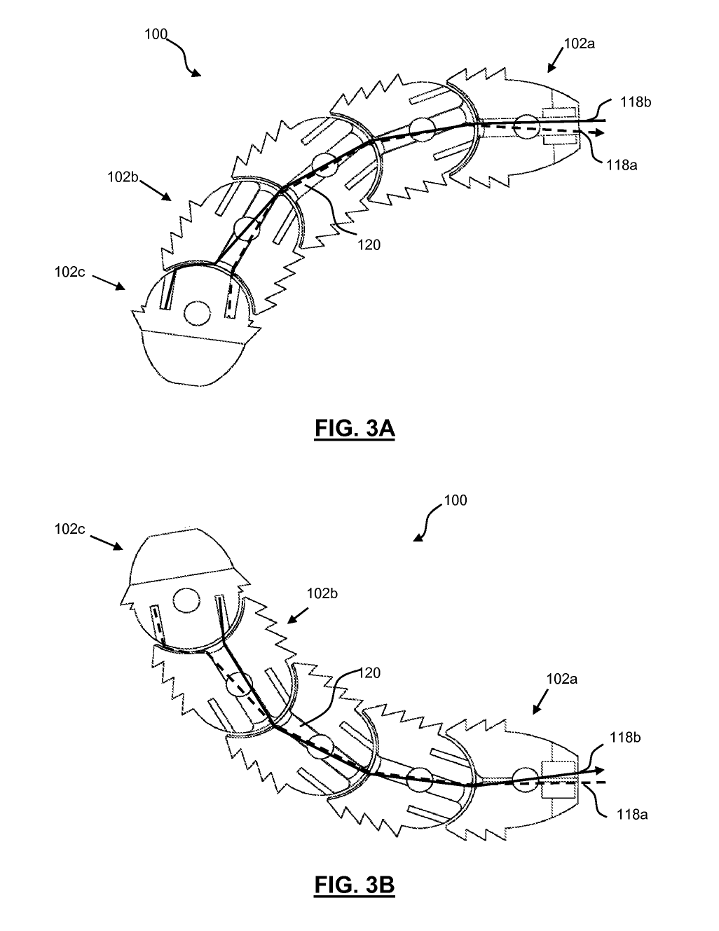

FIGS. 3A and 3B are sectional views showing one embodiment of a steering feature of the implant 100 using a plurality of tension members 118. While only tension members 118a and 118b are shown attached to the distal link 102c, there may be other tension members for the other links. The links include a channel 120 for the tension members to go through from the link 102c to the proximal end of the implant 100, exiting through one of the apertures 116b of proximal link 102a. It should be understood that each of the links 102b and 102c are coupled to at least two tension members 118 to control their rotation. The tension members are attached to each link on opposite sides of the pin 104 such that pulling on one of the tension members rotates the link in a clockwise direction and the pulling the other rotates the link in the counter clockwise direction. This allows the links to be rotated separately or together and steer the implant 100 in the desired direction. In FIG. 3A, when tension member 118a is pulled, link 102c is rotated in a counterclockwise direction, steering the implant in a downward direction. In the example shown in FIG. 3B, tension member 118b is pulled, rotating link 102c in a clockwise direction, steering the implant in an upward direction.

The top portion 106 and the bottom portion 108 may include a plurality of protrusions or teeth 122 (hereinafter, referred to as “teeth”). The teeth 122 may be spaced throughout the top portion 106 and the bottom portion 108 and are positioned so as not to interfere with the rotating of the links. As can be understood by one skilled, the teeth 122 can be configured to have variable thickness, height, and width as well as angles of orientation. The teeth 120 can be further configured to provide additional support after the steerable implant 100 is implanted in the vertebrae of the patient. The teeth 122 can reduce movement of the steerable implant 100 in the vertebrae and create additional friction between the vertebrae and the steerable implant 100. In the embodiment shown, the teeth 122 have a shape of triangular protrusions extending away from the surfaces of the top and bottom portions of the steerable implant 100 in a saw-tooth configuration. As can be understood by one skilled in the art, the teeth 122 can be configured to have any shape, size, and/or angular or any other orientation as well and can protrude any distance away from the surfaces of the steerable implant and can have any distance between them. In some embodiments, the tooth patterns have a quad-directional configuration (i.e., teeth 122 are facing in four different directions).

In some embodiments, the links 102 may have openings 103 configured to allow graft and Demineralized Bone Matrix (“DBM”) packing.

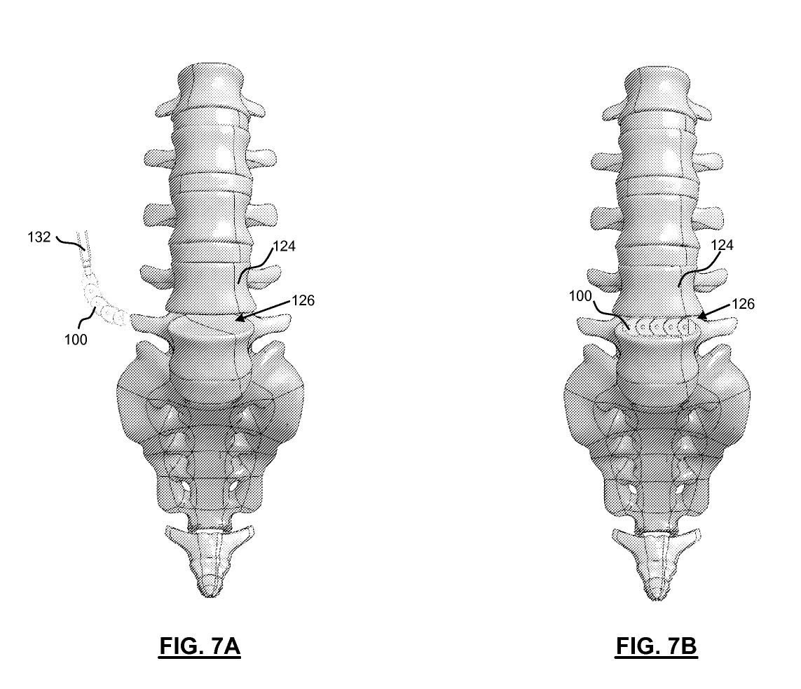

The rotation of the links 102 allow better movement and flexibility of the steerable implant 100 to match the shape of the vertebrae discs of the patient. As shown in FIGS. 7A and 7B, rotating the links in the caudal/cephalic direction allows the steerable implant 100 to be inserted into disk spaces that non-steering implants may have difficulty reach. As can be understood by one skilled in the art, the links 102 may have varying heights. For example, the height distal link 102c may be less than the height of intermediate 102b or proximal link 102a. Further, in some embodiments, the height of the links 102 can vary throughout the device. In other embodiments, the height can also vary between each side of the link 102. This means that, for example, a portion of the front side 110 can have a lesser height than another portion of the back side 110. Such variation in heights throughout the sides of the steerable implant 100 can be based on a particular design choice and further configured to accommodate various dimensions of the vertebrae of the patient.

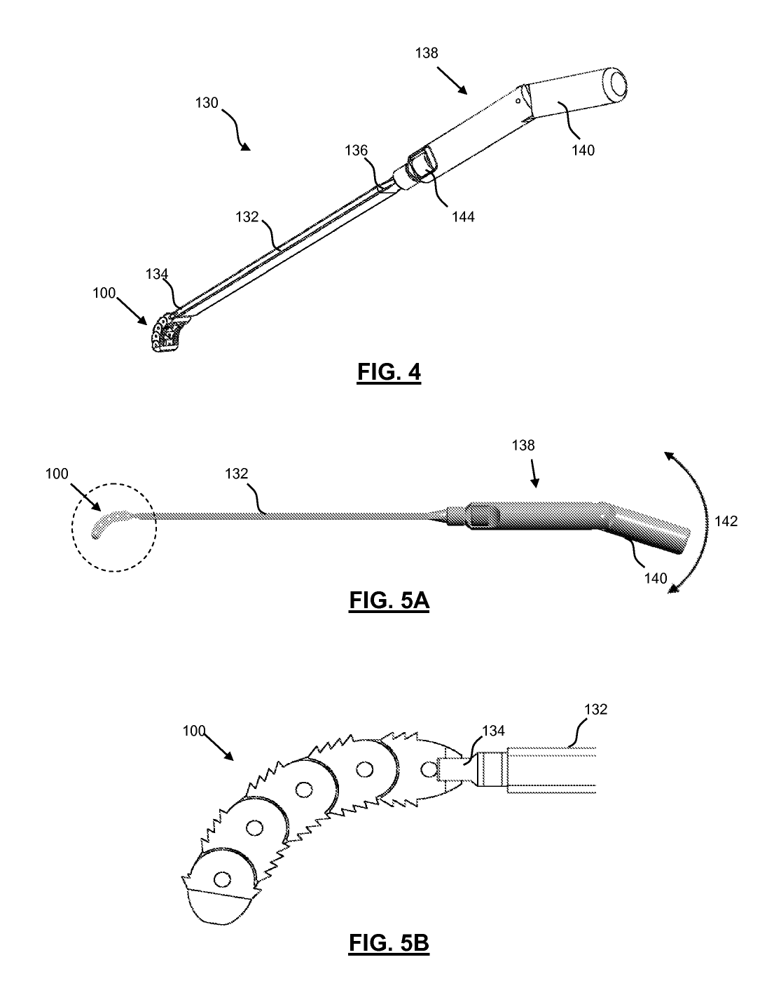

FIGS. 4, 5A, and 5B show one embodiment of an instrument 130 that may be used to implant the steerable implant 100. The instrument includes a shaft portion 132 having a distal end 134 that is coupled to the proximal end 112a of the implant 100 at apertures 116 and a proximal end 136 coupled to a handle 138 having a steering lever 140. The tension members 118A and 118B extend through the shaft 132 and handle 138 and are coupled to the steering lever 140. In use, by moving the steering lever 140 up or down (arrow 142), the implant links 102 move in either a clockwise or counterclockwise direction. There may also be various knobs 144 that used to attach the instrument 130 to the implant using attachment shafts or components not shown, or the knobs 144 may be used to disassemble the instrument 130.

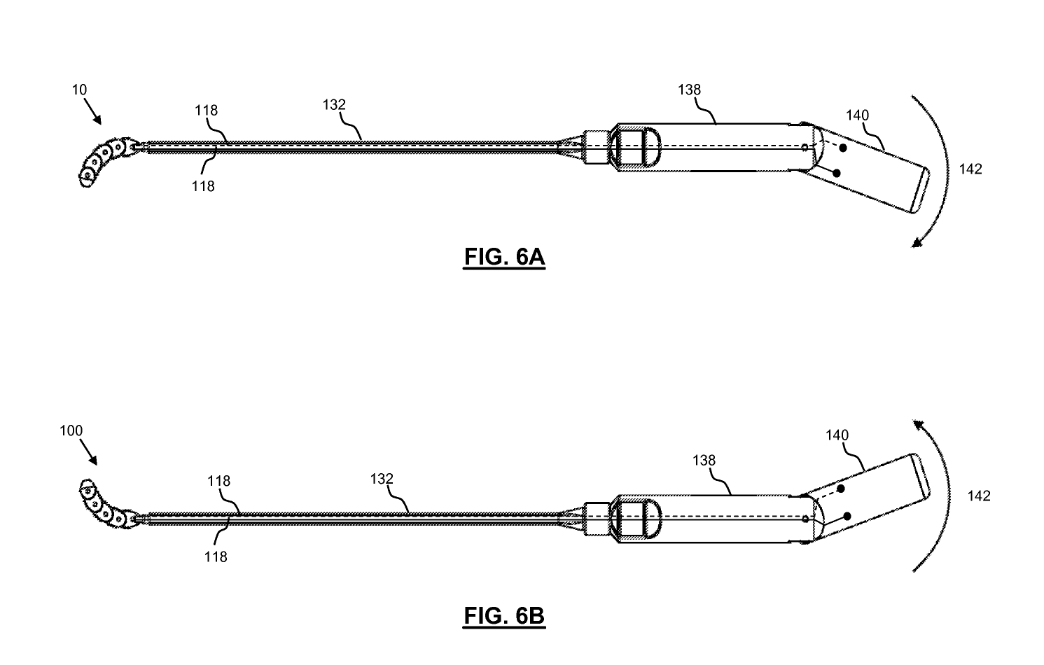

FIGS. 6A and 6B are sectional views illustrating steering of the implant 100 using the instrument 130 and actuation lever 140. Note, not all of the components are shown in these illustrations. In FIG. 6A, when the actuation lever 140 is moved in a downward direction 142a, tension member 118a (dashed line), is pulled, rotating the links 102 in a counterclockwise direction and steering the implant down. In FIG. 6B, when the actuation lever 140 is moved in an upward direction 142b, tension member 118b (solid line) is pulled, rotating the links 102 in a clockwise direction and steering the implant up. In the embodiments shown, the tension members 118a and 118b are attached to the links such that all of the links rotate or move in unison. In other embodiments, each of the links 112 may be attached to separate tension members 118 such that each of the links 112 may rotate or move separately. This may allow additional steering capability for the steerable implant 100.

FIGS. 7A and 7B illustrate installation of the steerable implant 100 into patient's vertebrae 124. FIG. 7A illustrates the steerable implant 100 being in a curved configuration for steering into the spinal opening 126. FIG. 7B illustrates the steerable implant 100 fully inserted between the vertebrae 124.

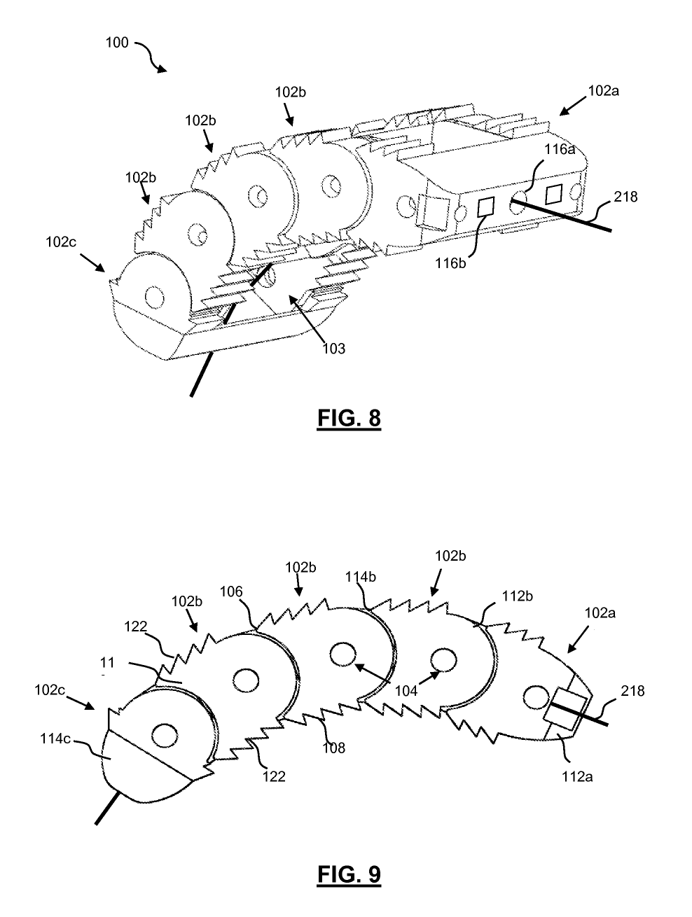

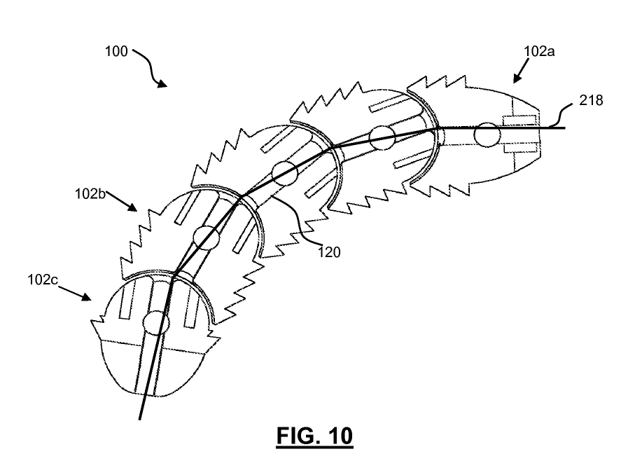

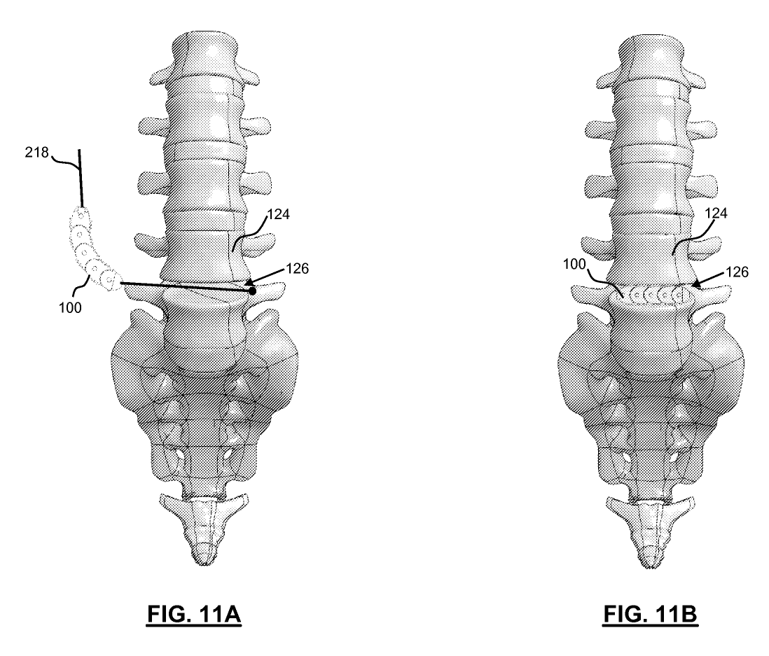

FIGS. 8-11B illustrate an embodiment of a steerable spine implant system in which the steerable spine implant 100 travels along a guide wire 218. For example, in some surgical procedures, the guide wire 218 may be inserted into the spinal opening 126 to enable insertion of various instruments including dilators, discectomy tools, and suction devices. The guide wire 218 may also be threaded through one of the various apertures 116, such as aperture 116a of the implant 100. When the links 102 are coupled together, the apertures 116 form a continuous guide wire channel from a proximal end to the distal end of the implant 100. The implant 100 then slides along the guide wire 218 into the spinal opening 126 and the guide wire 218 may subsequently be released. In this embodiment, the tension member 118 may be used in conjunction with the guide wire 218 or may be omitted.

In some embodiments, the steerable implant 100 can be manufactured from a biologically accepted inert material, such as PEEK (Polyetheretherketone) or metal. The steerable implant can be configured to be implanted between the vertebrae for treating degenerative or ruptured discs and/or for replacing damaged vertebral bodies. Each link can be particularly shaped and sized for its particular application.

In some embodiments, the steerable implant 100 can be sized larger than the vertebral body and/or configured to be implanted so that it rests on an apophyseal ring of a vertebrae (which is one of the strongest portions in a vertebral body). As can be understood by one skilled in the art, the steerable implant 100 can be sized and shaped as well as implanted as desired in accordance with a particular medical necessity or other factors.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A steerable spine implant, comprising:
a plurality of links rotatably coupled together at a hinge point including a proximal link, one or more intermediate links, and a distal link;
a plurality of tension members coupled to the intermediate and distal links, each link being coupled with first and second tension members on opposite sides of the hinge point,
wherein activating the first tension member rotates the plurality of links in a clockwise direction and activating the second tension member rotates the plurality of links in a counterclockwise direction.

2. The steerable implant according to claim 1, wherein the links further include a central channel sized to carry the plurality of tension members.

3. The steerable implant according to claim 2, wherein the tension members exit a channel at a proximal portion of the proximal link.

4. The steerable implant according to claim 1, wherein the links further include a top portion and a bottom portion having a plurality of protrusions or teeth.

5. The steerable implant according to claim 4, wherein the protrusions are configured to prevent movement of the steerable implant once the steerable implant is implanted.

6. The steerable implant according to claim 1, wherein the proximal link includes one or more adapter features or apertures configured to couple to one or more instruments.

7. The steerable implant according to claim 1, wherein one or more of the plurality of links includes openings for a graft or a DBM.

8. The steerable implant according to claim 1, wherein the distal link includes a tapered or shaped portion configured for insertion into a spinal area.

9. The steerable implant according to claim 1, wherein activating the first or second tension member comprises pulling the tension member proximally.

10. The steerable implant according to claim 1, wherein at least one of the plurality of links includes at least one of a varying height, length, thickness, and lordosis angle.

11. The steerable implant according to claim 1, wherein the plurality of links of the steerable implant comprises a biologically inert material.

12. The steerable implant according to claim 11, wherein two or more of the plurality of links comprise two or more different biologically inert materials.

13. A steerable spine implant system, comprising:
a guide wire; and
a plurality of links rotatably coupled together at a hinge point including a proximal link, one or more intermediate links, and a distal link, each of the plurality of links include an aperture sized to slide over the guide wire, wherein when the plurality of links are coupled together, the apertures form a continuous guide wire channel from a proximal end to a distal end of the implant.

14. The steerable implant according to claim 13, wherein the links further include a top portion and a bottom portion having a plurality of protrusions or teeth.

15. The steerable implant according to claim 14, wherein the protrusions are configured to prevent movement of the steerable implant once the steerable implant is implanted.

16. The steerable implant according to claim 13, wherein the proximal link includes one or more adapter features or apertures configured to couple to one or more instruments.

17. The steerable implant according to claim 13, wherein one or more of the plurality of links includes openings for a graft or a DBM.

18. The steerable implant according to claim 13, wherein the distal link includes a tapered or shaped portion configured for insertion into a spinal area.

19. The steerable implant according to claim 13, wherein at least one of the plurality of links includes at least one of a varying height, length, thickness, and lordosis angle.

20. The steerable implant according to claim 13, wherein the plurality of links of the steerable implant comprise a biologically inert material.
